(19) United States
(12) Patent Application Publication
(10) Pub. No.: US 2009/0023817 A1
(43) Pub. Date: Jan. 22, 2009

(54) Aqueous compositions comprising alkyl polyethylene glycol ether sulfates

(75) Inventors: Hendrik Ahrens, Kriflel (DE); Guillermo Maier, Haiming (DE); Klaus Raab, Burgkirchen (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 12/217,938

(22) Filed: Jul. 10, 2008

(30) Foreign Application Priority Data
Jul. 13, 2007 (DE) 102007032670.1

(51) Int. Cl. A61K 8/86; C11D 17/00; C08L 81/00; B01F 17/52

(52) U.S. Cl. 514/772.3; 516/28; 510/337; 524/609

(57) ABSTRACT

The present invention provides aqueous compositions comprising, as well as water, 75% to 90% by weight of alkyl polyethylene glycol ether sulfates which are a mixture of compounds of formula (1)

R¹—O—(CH₂—CH₂—O)ₓ—SO₃M (1)

where
R¹ is a branched alkyl or alkenyl group having 8 to 14 carbon atoms and at least one tertiary carbon atom,
x is from 3 to 15,
M is an alkali metal ion or an ammonium ion,
   with compounds of formula (2)

R²—O—(CH₂—CH₂—O)_{y}—SO₃M′ (2)

where
R² is a linear alkyl or alkenyl group having 8 to 14 carbon atoms,
y is from 3 to 15,
M′ is an alkali metal ion or an ammonium ion,
   wherein the mixing ratio of compounds of formulae (1) and (2) is chosen such that the compounds of formula (1) comprise at least 10% by weight and at most 99% by weight of the total amount of compounds of formulae (1) and (2).

The present invention is described in the German priority application No. 10 2007 032 670.1, filed Jul. 13, 2007, which is hereby incorporated by reference as is fully disclosed herein.

The present invention provides aqueous, flowable compositions comprising alkyl polyethylene glycol ether sulfates with an active ingredient content of more than 75% by weight, a process for their production and their use.

Smooth production and processing of aqueous compositions of alkyl polyethylene glycol ether sulfates with a high active ingredient content is often hindered by the fact that these solutions are very viscous and not very flowable. Aqueous compositions of alkyl polyethylene glycol ether sulfates are generally flowable at room temperature up to an active ingredient content of about 30% by weight and display viscosities of up to 500 mPa·s, typically up to 100 mPa·s. A Brookfield viscometer is a useful instrument for measuring the viscosity of such compositions.

The viscosity of more highly concentrated aqueous compositions of alkyl polyethylene glycol ether sulfates with an active ingredient content of more than 30% by weight is temperature dependent, so that these compositions can be made to flow by heating, but the heating of such compositions is associated with problems. Heatable containers are not universally available. Alkyl polyethylene glycol ether sulfates tend to hydrolyze at higher temperatures, so that the ether sulfate bond is split and the active concentration decreases.

Viscosity can also be reduced by addition of certain materials. WO—91/02045 describes the addition of oleic acid sulfonates and other components as viscosity regulators to aqueous anionic surfactant concentrates comprising alkyl polyethylene glycol ether sulfates. U.S. Pat. No. 4,191,704 describes controlling the viscosity by neutralizing with alkylamines and alkanolamines instead of aqueous sodium hydroxide solution whereby the resulting neutralizate is flowable even at active ingredient contents of more than 60% by weight.

The process for producing aqueous compositions of alkyl polyethylene glycol ether sulfates is described in U.S. Pat. No. 2,644,831, U.S. Pat. No. 2,654,772, U.S. Pat. No. 2,758,977 and U.S. Pat. No. 2,214,254.

A further process for producing alkyl polyethylene glycol ether sulfates by means of sulfur trioxide is described in WO—91/05764.

The production of aqueous concentrates of fatty alcohol ether sulfate alkaline earth metal salts having a solids content of 50% to 75% by weight is described in DE-A-44 46 363.

Alkyl polyethylene glycol ether sulfates are used as surfactants for example in the laundry detergent and cosmetics industry, in industrial, institution and household cleaners, as dispersing and wetting agents for example in pigment dispersions or as emulsifiers in emulsion polymerization.

It is an object of the present invention to provide concentrated aqueous compositions of alkyl polyethylene glycol ether sulfates having an active ingredient content of more than 75% by weight, sufficient flowability at room temperature and simple metering properties. The distinctly reduced water content, compared with the prior art, and the increased active ingredient content reduce transportation costs per unit weight of active ingredient and make it possible to omit the addition of preservatives which interferes with some applications.

We have found that alkali metal or ammonium salts of branched alkyl polyethylene glycol ether sulfates or mixtures of branched and linear alkyl polyethylene glycol ether sulfates form flowable, efficiently meterable and pumpable solutions at an active ingredient content between 75% and 90% by weight in aqueous compositions.

The present invention accordingly provides aqueous compositions comprising, as well as water, 75% to 90% by weight of alkyl polyethylene glycol ether sulfates which are a mixture of compounds of formula (1)

R¹—O—(CH₂—CH₂—O)ₓ—SO₃M (1)
where
R¹ is a branched alkyl or alkenyl group having 8 to 14 carbon atoms and at least one tertiary carbon atom,
x is from 3 to 15,
M is an alkali metal ion or an ammonium ion,
with compounds of formula (2)

R²—O—(CH₂—CH₂—O)_{y}—SO₃M′ (2)
where
R² is a linear alkyl or alkenyl group having 8 to 14 carbon atoms,
y is from 3 to 15,
M′ is an alkali metal ion or an ammonium ion,
wherein the mixing ratio of compounds of formulae (1) and (2) is chosen such that the compounds of formula (1) comprise at least 10% by weight and at most 99%, preferably at least 20% and at most 90%, in particular at least 30 and at most 80%, by weight of the total amount of compounds of formulae (1) and (2).

In the compounds of formulae (1) and (2), R¹ and R² are each preferably primary alkyl or alkenyl radicals, i.e., radicals attached to the oxygen atom via a primary carbon atom.

In formula (1), R¹ is preferably a branched alkyl group having 9 to 13 and in particular 10 to 12 carbon atoms and at least one tertiary carbon atom. R¹ in a further preferred embodiment contains 1 to 5 tertiary carbon atoms.
x is preferably from 5 to 10.
M is preferably sodium.

In formula (2), R² is preferably a linear alkyl group having 9 to 13 and in particular 10 to 12 carbon atoms.
y is preferably from 5 to 10.
M′ is preferably sodium.

x and y each represent the average number of oxethylene units when the poly(oxethylene) units of formulae (CH₂—CH₂—O)ₓ and (CH₂—CH₂—O)_{y} of the compounds of formulae (1) and (2) do not have unitary lengths.

In a preferred embodiment, the compositions of the present invention comprise 75% to 90% by weight and in particular 80% to 88% by weight of compounds of formula (1) and, if present, of formula (2).

In a further preferred embodiment, the compositions of the present invention comprise 5% to 25% by weight and in particular 6% to 20% by weight of water. In a further preferred embodiment, the compositions of the present invention comprise water ad 100% by weight.

The production of alkali metal salts of alkyl polyethylene glycol ether sulfates of primary alcohols typically generates secondary components, such as alkali metal sulfates, alkali metal chlorides or other alkali metal salts and nonsulfated portions such as polyethylene glycols, alkyl polyethylene glycols and alcohols of formulae R¹—OH and R²—OH, which secondary components may be present in the compositions of the present invention at up to 5% by weight, generally 0.5 to 4% by weight.

In a further preferred embodiment, the compositions of the present invention may contain in total up to 3% by weight, preferably 0.1% to 2% by weight of additives such as pH buffers, for example sodium carbonate, sodium bicarbonate or sodium citrate, solvents or viscosity regulators, as a result of admixture. The admixture of preservatives to control the growth of germs in the aqueous compositions of the present invention is possible, but not essential and may be omitted because of the high level of alkali metal or ammonium alkyl polyethylene glycol ether sulfates (active ingredient content) and because of the low water content.

The active ingredient content of the compositions can be determined by the method described in the ISO 2271 standard.

The compositions according to the present invention are produced by reacting alkyl polyethylene glycols of formula R¹—O—(CH₂—CH₂—O)ₓ—H or mixtures of alkyl polyethylene glycols of formulae R¹—O—(CH₂—CH₂—O)ₓ—H and R²—O—(CH₂—CH₂—O)_{y}—H with SO₃ or some other sulfating agent such as for example oleum, amidosulfonic acid or chlorosulfonic acid. The preferred sulfating agent is SO₃, in particular gaseous SO₃, which may be diluted with an inert gas such as, for example, air or nitrogen. The amount of SO₃ or of some other sulfating agent used per 1 mol of alkyl polyethylene glycol is in the range from 0.9 to 1.3 mol and in particular in the range from 1.0 to 1.1 mol. The resulting acid sulfuric esters of the formula R¹—O—(CH₂—CH₂—O)ₓ—SO₃H or R²—O—(CH₂—CH₂—O)_{y}—SO₃H are neutralized with bases such as, for example, alkali metal hydroxides or ammonia, preferably with the concentrated aqueous solutions of these bases, in particular with aqueous sodium hydroxide solution, ideally immediately after formation of the acid sulfuric esters with good commixing and cooling. The amount of base required is determined by acid-base titration of a sample of the resulting acid sulfuric ester or by continuous on-line potential measurement. Depending on the pH value desired for the composition of the present invention, from 0.99 to 1.1 mol of base are generally used per 1 mol of SO₃ used. The preferred base is 50% strength aqueous sodium hydroxide solution. The neutralization temperature is in the range from 30 to 80° C., preferably 40 to 60° C. The reaction with the SO₃ and the neutralization are both preferably carried out in a continuous mode. The water content can be calculated approximately from the water formed in the neutralization and from the water content of the aqueous base, and can be accurately determined by Karl Fischer titration. After addition of further water if required and addition of the desired admixtures, for example buffer to set a certain pH value, for example in the range of pH 6 to 8, the present concentrated aqueous compositions of alkyl polyethylene glycol ether sulfates are obtained.

The process leads to compositions that are flowable at 25° C. In a preferred embodiment, the compositions obtained have a viscosity of at most 5,000 mPa—s, i.e., they are flowable.

Compared with hitherto available alkyl polyethylene glycol ether sulfates, the present high-concentrated compositions of alkyl polyethylene glycol ether sulfate and water have the advantage that no preservative is required. This is an advantage when the compositions of the present invention find use as emulsifiers in emulsion polymerization and the polymer dispersions produced therewith are used in food contact applications in which only few preservatives are permissible, for example as binders and film-formers for coating packages, paper, metals and for adhesives.

The low water content is a further advantage of compositions according to the present invention. There are some applications where the relatively large amount of water present in prior art compositions is unwelcome. To obtain liquid, flowable products, the previously available solutions of alkyl polyethylene glycol ether sulfates were adjusted to an active ingredient content of about 30%. The difference consisted substantially of water. High-concentrated pigment formulations are preferably manufactured using high-concentrated dispersants. In the case of water-diluted dispersants, the pigment formulation obtained is unintentionally diluted. The compositions according to the present invention are by virtue of their low water content very useful as dispersants for high-concentrated pigment formulations.

### EXAMPLE 1

An alkyl polyethylene glycol was produced by ethoxylation of a primary alcohol whose alkyl chain was about 50% singly branched carbon chains and about 50% linear carbon chains and whose average carbon chain length was 11 carbon atoms, with ethylene oxide in a molar ratio of 1:7 for primary alcohol to ethylene oxide. This alkyl polyethylene glycol having an average degree of ethoxylation of 7 ethylene glycol units was continuously reacted at 50° C. with an equimolar amount of sulfur trioxide to form the corresponding acid sulfuric ester. 500 g of this liquid acid sulfuric ester and 73.5 g of 50% aqueous sodium hydroxide solution were metered conjointly, from two dropping funnels, into a close-clearance stirred vessel cooled to 40-46° C. Stirring should not be too rapid, or excessive foam will be generated. A little aqueous sodium carbonate solution was used to set pH 8. The pH was measured after drawing a small aliquot which had been diluted with water to an active ingredient content of 1% for the purposes of the pH measurement only.

The composition, which was liquid at 20° C., had the following composition:
about 86% of sodium alkyl polyethylene glycol ether sulfate with 7 ethylene glycol units (reckoned from Epton titration assuming an average molar mass of 577 g/mol)
about 9% of water (Karl Fischer titration)
about 0.7% of sodium sulfate
about 2.7% of unconverted alkyl polyethylene glycol ether (determination of neutral part).

### EXAMPLE 2

Example 1 was repeated on production plant scale, with increased quantities, leading to a room temperature liquid composition having the following composition:
about 86% of sodium alkyl polyethylene glycol ether sulfate with 7 ethylene glycol units
about 12% of water
about 1% of sodium sulfate
about 1% of unconverted alkyl polyethylene glycol ether.

The composition displayed a pH of 7.6 (measured in water at 1% active ingredient) and a Brookfield viscosity at 25° C. of about 200 mPa·s.

### EXAMPLE 3

A liquid 30% aqueous solution of sodium alkyl polyethylene glycol ether sulfate having on average about 7 ethylene glycol units and having an alkyl chain which was 47% various branched C₁₁H₂₃ groups, 47% a straight-chain C₁₁H₂₃ group and 6% C₁₀H₂₁ and C₁₂H₂₅ groups was very slowly metered into a rotary evaporator at 30 to 50° C. and a pressure of about 5 mbar. The water was distilled off under vigorous foaming until a residual water content of 1.3% (Karl Fischer titration) and an active ingredient content of 94% (Epton titration) had been reached. This intermediate product was solid, non-flowable at 20° C. and even at 40° C. and had a drop point of 85° C., and became on addition and mechanical incorporation of an additional 7.4% of water a 20° C. liquid and flowable composition having an active ingredient content of 87.5%.

### COMPARATIVE EXAMPLE

A liquid dilute aqueous solution of sodium alkyl bis(ethylene glycol) ether sulfate having on average about two ethylene glycol units and having an alkyl chain which was 70% a straight-chain C₁₂H₂₅ group, 26% a straight-chain C₁₄H₂₉ group and 4% a straight-chain C₁₆H₃₃ group was very slowly metered into a rotary evaporator at 30 to 50° C. and a pressure of about 5 mbar. The water was distilled off under vigorous foaming until an active ingredient content of 92% (Epton titration) had been reached. Various amounts of water were added and mechanically incorporated to produce four mixtures having an active ingredient content of 90%, 85%, 80% and 75%, which were all firm, non-flowable pastes at 20° C.

Use Example 1 of producing a polymer dispersion with the inventive composition of Example 1

1800.0 g of a monomer emulsion consisting of 410.3 g of completely ion-free water, 23.5 g of the inventive composition of Example 1, 2.2 g of dodecyl mercaptan, 150.0 g of methyl methacrylate, 350.0 g of 2-ethylhexyl acrylate, 850.0 g of n-butyl acrylate and 14.0 g of methacrylic acid and also 57.0 g of an initiator solution consisting of 7.1 g of ammonium peroxodisulfate and 49.9 g of completely ion-free water are prepared.

A 3 liter reaction vessel is initially charged with 263.0 g of completely ion-free water which are heated to 80° C. under nitrogen on a water bath. Then, 17.0 g of the initiator solution are added and immediately the continuous addition of the 1800.0 g of monomer emulsion and the remaining 40.0 g of initiator solution is commenced. The two components are added over three hours under nitrogen with continuous stirring using an anchor stirrer. Thereafter, the reaction mixture is maintained at 80° C. for a further hour and subsequently cooled down to room temperature. The pH of the polymer dispersion obtained is adjusted to pH 7-8 with 12.5% ammonia solution.

The resulting polymer dispersion has a solids content of 65% and a coagulum content of<0.050% on a 100 μm sieve and of<0.080% on a 40 μm sieve.

Use Example 2 of producing a pigment formulation with the inventive composition of Example 1

To produce 150.0 g of a pigment formulation, the following components are initially charged to a grinding container and pre-dissolved with a dissolver disk.
17.4 g of the inventive composition of Example 1 as dispersant
30.0 g of propylene glycol
3.0 g of defoamer
0.3 g of preservative
31.8 g of completely ion-free water

Then, 67.5 g of the pulverulent pigment designated Color Index PR 112 are added and predispersed with the dissolver. Fine dispersion is done in a bead mill with zirconium mixed oxide beads of size d=1 mm with cooling. Subsequently, the grinding media are separated off and the pigment formulation is isolated.

A flowable, homogeneous pigment formulation is obtained after the grinding operation.

1. An aqueous composition comprising, water, and 75% to 90% by weight of alkyl polyethylene glycol ether sulfates which are a mixture of compounds of formula (1)
R¹—O—(CH₂—CH₂—O)ₓ—SO₃M (1)
wherein
R¹ is a branched alkyl or alkenyl group having 8 to 14 carbon atoms and at least one tertiary carbon atom,
x is from 3 to 15,
M is an alkali metal ion or an ammonium ion,
with compounds of formula (2)
R²—O—(CH₂—CH₂—O)_{y}—SO₃M′ (2)
wherein
R² is a linear alkyl or alkenyl group having 8 to 14 carbon atoms,
y is from 3 to 15,
M′ is an alkali metal ion or an ammonium ion,
wherein the mixing ratio of compounds of formulae (1) and (2) is selected such that the compounds of formula (1) comprise at least 10% by weight and at most 99% by weight of the total amount of compounds of formulae (1) and (2).

2. The aqueous composition as claimed in claim 1, wherein the mixing ratio of compounds of formulae (1) and (2) is selected such that the compounds of formula (1) comprise at least 20% by weight and at most 80% by weight of the total amount of compounds of formulae (1) and (2).

3. The aqueous composition as claimed in claim 1, wherein R¹ and R² are each primary alkyl or alkenyl radicals.

4. The aqueous composition as claimed in claim 1, comprising 80% to 88% by weight of compounds of formula (1) and of formula (2).

5. The aqueous composition as claimed in claim 1, comprising 6% to 20% by weight of water.

6. The aqueous composition as claimed in claim 1, wherein R¹ is a branched alkyl group having 9 to 13 carbon atoms and at least one tertiary carbon atom.

7. The aqueous composition as claimed in claim 1, wherein x is from 5 to 10.

8. The aqueous composition as claimed claim 1, wherein M is sodium.

9. The aqueous composition as claimed in claim 1, wherein R² is a linear alkyl group having 9 to 13 carbon atoms.

10. The aqueous composition as claimed in claim 2, wherein y is from 5 to 10.

11. The aqueous composition as claimed in claim 2, wherein M′ is sodium.

12. A process for producing an aqueous as claimed in claim 1, comprising the steps of reacting alkyl polyethylene glycols of the formula R¹—O—(CH₂—CH₂—O)ₓ—H or mixtures of alkyl polyethylene glycols of the formulae R¹—O—(CH₂—CH₂—O)ₓ—H and R²—O—(CH₂—CH₂—O)_{y}—H, with 0.9 to 1.3 mol of a sulfating agent to form acid sulfuric ester and neutralizing the acid sulfuric esters of the formulae R¹—O—(CH₂—CH₂—O)ₓ—SO₃H and R²—O—(CH₂—CH₂—O)_{y}—SO₃H with at least one base.

13. The process as claimed in claim 12, wherein the sulfating agent is SO₃, oleum, amidosulfonic acid or chlorosulfonic acid.

14. The process as claimed in claim 12, wherein the at least one base is an alkali metal hydroxide or ammonia.

15. The process as claimed in claim 12, wherein the neutralizing step occurs at a temperature of 30 to 80° C.

16. The process as claimed in claim 12, wherein, after the neutralizing step an amount of buffer is added so that the pH of the resulting composition is in the range from 6 to 8.

17. The process as claimed in claim 1, wherein the process is conducted in continuous mode operation.

18. A surfactant for laundry detergent and cosmetics industry, and in industrial, institution and household cleaners, comprising an aqueous composition as claimed in claimed in claim 1.

19. The aqueous composition as claimed in claim 1, wherein y is from 5 to 10.

20. The aqueous composition as claimed in claim 1, wherein M′ is sodium.

21. A dispersing or wetting agent in pigment dispersions comprising an aqueous composition as claimed in claim 1.

22. An emulsifier in emulsion polymerization comprising an aqueous composition as claimed in claim 1.
